(19) United States
(12) Patent Application Publication
(10) Pub. No.: US 2020/0306231 A1
(43) Pub. Date: Oct. 1, 2020

(54) ORAL HYPOGLYCEMIC AGENTS AS FOOD ADDITIVES AND SUPPLEMENTS

(71) Applicants: Vershalee Shukla, Phoenix, AZ (US); Pablo A. Prichard, Phoenix, AZ (US)

(72) Inventors: Vershalee Shukla, Phoenix, AZ (US); Pablo A. Prichard, Phoenix, AZ (US)

(21) Appl. No.: 16/759,051

(22) PCT Filed: Oct. 26, 2018
(86) PCT No.: PCT/US18/57782

§ 371 Date: Apr. 24, 2020

Related U.S. Application Data
(60) Provisional application No. 62/577,295, filed on Oct. 26, 2017.

(51) Int. Cl. A61K 31/4375; A23L 33/00; A23L 33/105; A23L 33/15; A23L 33/16; A61K 31/155

(52) U.S. Cl. CPC A61K 31/4375; A23L 33/30; A23L 33/105; A61K 9/0053; A23L 33/16; A61K 31/155; A23L 33/15

(57) ABSTRACT

Food compositions or dietary supplements comprising an effective amount of an additive that comprises at least one oral hypoglycemic agent for the treatment, prevention, control and management of blood sugar and insulin spikes associated with ingestion of food comprising sugars and simple carbohydrates. Methods for the use of oral hypoglycemic agents as additives during manufacture or preparation of food to lower the glycemic index of the food or glycemic response thereto are also described.

### FIELD OF THE INVENTION

The invention is directed to pre-packaged foods for human ingestion or consumption, including without limitation solid, baked and non-baked, powders, and liquids, which incorporate oral hypoglycemic additives, not heretofore utilized as food additives, to protect against blood sugar and insulin spikes known to be associated with high glycemic index foods The invention is also directed to use of oral hypoglycemic agents as additives at the food manufacture or preparation stage or as supplements self-initiated by consumers to incorporate into their food as protection against blood sugar and insulin spikes caused by diets that include high amounts of sugar and simple carbohydrates. The invention is further directed to a method for reducing glycemic index of food and for regulating blood sugar alterations or swings associated with conventional diets and/or high glycemic index foods. The hypoglycemic agents may be both prescription and non-prescription agents that are known to lower blood glucose levels and in turn stabilize insulin spikes, but that have not previously been utilized as food additives or dietary supplements.

### BACKGROUND OF THE INVENTION

The Centers for Disease Control (CDC) recently released 2016 state- and territory-specific data relating to the prevalence of obesity in the United States. The CDC's data reflects that all fifty states have more than 20% of adults with obesity. The CDC estimates vary across states, with rates ranging from 22.3% in Colorado to 37.7% in West Virginia, Geographically, the South had the highest prevalence of obesity, the CDC report found, while the lowest prevalence was in the West, at 26%. The data indicated that adults with more education were less likely to report being obese. Individuals without a high school education had the highest self-reported obesity, at 35.5%, while the rates for high school graduates, adults with some college, and college graduates were 32.3%, 31%, and 22.2%, respectively.

Obesity puts millions of Americans at risk for serious chronic diseases and health conditions, including type 2 diabetes and conditions associated therewith, cardiovascular disease, stroke, certain cancers, poorer mental health, premature aging, and infertility and problems with pregnancy. Obesity and the risk for chronic disease and other health conditions is a global problem.

The CDC also estimates that 33 million Americans have diabetes mellitus and 84 million are either pre-diabetic or glucose intolerant/insulin resistant. The sequelae of diabetes in general include coronary artery disease, peripheral vascular disease, peripheral neuropathy, renal nephropathy, retinopathy, renal failure, and hypertension, among others. Insulin resistance is a condition in which the cells fail to respond to insulin properly. Insulin resistance also leads to immunosuppression and increased risks of many cancers, including without limitation liver, breast, pancreas, and endometrial.

Diabetes and pre-diabetic conditions are becoming more prevalent in young Americans with 1.5 million Americans aged 18-44 diagnosed with diabetes mellitus Type 2, or insulin resistant type, diabetes. As the disease progresses, patients may experience a relative lack of insulin, necessitating addition of insulin to the treatment regimen. Insulin increases the costs of diabetes treatment significantly.

Global health expenditures for diabetes mellitus were 673 billion in 2015 according to the International Diabetes Federation. Approximately one-fifth (⅕) of American health care expenditures are related to diabetes according to the American Diabetes Association.

One of the risk factors for diabetes, among others, is diet. In a fast-paced society, many individuals simply do not take the time to plan and consume healthy diets and snacks. As one example, the American diet traditionally includes, in large portion, high glycemic index foods containing sugars and simple carbohydrates that are readily converted into simple sugars, which cause a dramatic increase in blood glucose and insulin spikes. Certain cultures also have high glycemic index foods in their diets, which include a variety of sugars and starches that also contribute to increases in blood glucose and insulin spikes. Still, even when instructed to avoid high glycemic index foods, some individuals may not be able to afford to eat a healthier diet and rely on less expensive foods that have a high concentration of sugars and simple carbohydrates.

Repeated episodes of increased blood glucose and insulin spikes over time contribute greatly to insulin resistance and diabetes mellitus and all its inherent sequelae. Diabetes and pre-diabetic conditions traditionally are treated by diet, exercise, and medication. However, this is usually met with resistance in some patient populations as compliance with diet, exercise and even medication regimens is poor for many reasons. Patients are reluctant to give up their normal diet, and many are forgetful and do not adhere to medication regimens or are inconsistent in adherence. Some patients faced with increasing amounts of medication costs are especially erratic with adherence.

Reluctance to adhere to a diet, exercise and medication regimen is especially problematic in patients who are not yet diagnosed with diabetes mellitus but are at high risk for developing it due to lifestyle, family history, and diet. Since diabetes mellitus is a very slow progressive disease (known as the ‘silent killer’), patients do not feel ill until the disease is very advanced. Thus, compliance with diet, exercise and medication regimens in the pre-diabetic and early stages of the disease is quite limited.

Initially, patients with pre-diabetes may be pro-active and self-initiate exercise, a healthier diet or supplements, but their motivation tends to waver as busy lifestyles make it difficult to remember to take supplements with meals, carry around pills, or find low sugar and carbohydrate diets (lower glycemic index foods) on the run.

Glycemic index (GI) of a food is the blood glucose response to a food, as defined by a comparison of the blood glucose response of a food to that of a reference food or control (with the same amount of available carbohydrate). Glycemic index is a ranking of carbohydrates on a scale of from 0 to 100 according to the extent to which they raise blood sugar (glucose) levels after eating. Foods with a high glycemic index are rapidly digested, absorbed and metabolized and result in marked fluctuations in blood sugar levels.

A low-glycemic index food causes blood levels to increase more slowly and steadily, It is well-known that lower glycemic index foods have health benefits. In efforts to lower the glycemic index of foods and the effects on the body, work has been undertaken to alter the carbohydrates and sugars of food stuffs through processing or pretreating raw materials to lower sugar content.

For example, attempts to reduce digestibility and thus glycemic index of starch by chemical or physical modification or by encapsulation or coating have been described in U.S. Pat. Nos. 5,246,723 and 5,695,803, and EP 0749697, among others. Another method of lowering absorption of sugar from the intestines is by delaying digestion of carbohydrates (sugars and starches) through administration of an effective amount of one or more flavonoids to an animal or human in conjunction with food as described in U.S. Patent Publication No. 20120122806, now U.S. Pat. No. 8,865,661.

Replacement of high glycemic index carbohydrates in food with ingredients having a lower glycemic index and use of high viscosity dietary fibers has also been utilized to reduce the glycemic index of food. One such approach to lower the glycemic index of food is direct incorporation of alpha-cyclodextrin in foods as a substitute for other sugars foods during manufacture or preparation as described in U.S. Patent Publication No. 2004/0161526. Another approach is the addition of water-soluble dietary fibers having high viscosity, which are stated to lower absorption of glucose from food as described in U.S. Patent Publication No. 20020012733.

Another method of reducing glycemic response to carbohydrate foods is by the use of a pre-mixed, flour-containing mixture of pulverized edible parts of Okra plant species with pulverized edible Vigna plant species and one or more types of food-based carbohydrates selected from cereals or non-cereals for the preparation of food as described in WO 2010077127 A1.

The foregoing efforts are not without disadvantages, such as increased cost for food or supplements, negative effects on the taste or texture of food or its nutrient value, manufacturing difficulties of incorporating certain ingredients, or insufficient reduction of glycemic index, More work is needed to control the glycemic index of foods and/or regulate the effects of ingested sugar and carbohydrates on blood sugar and insulin spikes.

There remains a need for methods to address the continuing increase in diabetes mellitus prevalence worldwide. While diet, exercise and medication regimens will likely remain the mainstay of prevention and treatment, it is clear that these regimens and other attempts to bolster them are not enough.

It has been discovered that direct addition of certain oral hypoglycemic agents to food has a significant effect on blood sugar levels and insulin spikes associated with ingestion of foods containing sugar and simple carbohydrates, hence lowering their glycemic index. The present invention focuses on direct addition of oral hypoglycemic agents to food, without the need for expensive ingredients or food processing or treatment to lower the sugar content of food, or expensive ingredients, medications or supplements to delay digestion and prevent sugar absorption. The invention is directed to use of oral hypoglycemic agents, not previously utilized as food additives or in dietary supplements. Further, addition of the oral hypoglycemic agents of the invention to nutraceuticals and other dietary supplements already known to reduce blood sugar or support blood sugar homeostasis may also be beneficial and is within the scope of the invention.

There are a number of both prescription and non-prescription oral hypoglycemic agents on the market, as well as agents that potentiate the effects of oral hypoglycemic agents. Of the prescription oral hypoglycemic agents, none have previously been utilized or considered for use in food or as a dietary supplement to reduce glycemic index of food or reduce blood glucose and insulin spikes associated with high glycemic index foods. Similarly, many non-prescription oral hypoglycemic agents, while believed to support blood sugar homeostasis, have not been utilized or considered for use in food at the manufacture or preparation stage.

As discussed herein, the oral hypoglycemic agents of the invention have a multitude of health benefits including without limitation reducing blood glucose and insulin spikes, facilitating weight/fat loss, delaying the aging process, as well as having antibacterial and anti-inflammatory and immune-enhancing properties. It is well known that obesity creates a risk for serious chronic diseases and health conditions, including type 2 diabetes and conditions associated therewith, dyslipidemias, high blood pressure, cardiovascular disease, stroke, certain cancers, poorer mental health, premature aging, and infertility and problems with pregnancy, Use of the oral hypoglycemic agents of the invention, which have not previously been utilized as food additives or ingredients or as dietary supplements, provide yet another approach for addressing obesity and the risk for chronic disease and other health conditions that continue to be global problems.

In addition to combatting the aforenoted problems associated with heavy use and reliance on high glycemic index foods, using the oral hypoglycemic agents of the invention as additives in foods at the manufacture or preparation stage aims at dramatically increasing compliance with supplements or medication regimens. Using oral hypoglycemic agents in combination with other dietary supplements may also be of value to achieve compliance or adherence with medication regimens. Accordingly, the oral hypoglycemic agents of the invention may be used alone or in combination with other components known to lower blood sugar or that have an effect on blood sugar homeostasis, to take advantage of synergistic hypoglycemic effects.

The present invention is directed to use of oral hypoglycemic agents as food additives at the manufacture or preparation stage or as food additives for use by consumers in self-initiated dietary regimens, which have not heretofore been utilized as food additives or dietary supplements despite having known blood glucose reducing effects. By way of example, the invention contemplates use of biguanides, sulfonylureas, thiazolidinediones (TZD's), alpha-glucosidase inhibitors, dipeptidyl-peptidase-4 (DPP-4) inhibitors, and sodium glucose cotransporter 2 (SGLT2) inhibitors as food additives and dietary supplements.

One oral hypoglycemic agent preferably used in the present invention is metformin, although the invention is not limited to metformin. Metformin is FDA-approved as a treatment for Type 2 diabetes and is utilized in polycystic ovarian syndrome, prevention of Type 2 diabetes in patients with impaired fasting glucose or glucose tolerance tests (pre-diabetes) and treatment of gestational diabetes mellitus, although these latter uses are not FDA-approved. Metformin is a prescription medication that is used to increase the body's response to insulin on a cellular level and is useful in both pre-diabetes and diabetes treatment. Metformin has an excellent track record for safety and has few adverse effects, thus making it an ideal additive to reduce the increase in blood sugar and insulin spikes associated with ingestion of high carbohydrate and sugar containing foods. For purposes of this invention, metformin as well as other oral hypoglycemic agents may be used alone or in combination with other oral hypoglycemic agents, both prescription and non-prescription, to reduce further the glycemic index of food.

The invention contemplates the use of oral hypoglycemic agents as additives to a variety of food stuffs, including without limitation, including without limitation, solid and liquid food and beverages and snack foods.

It is an object of the invention to provide oral hypoglycemic compositions that may be utilized as food additives for food sources at the preparation or production stage or self-initiated consumer regimens, to reduce the glycemic index of the food and subsequent blood glucose and insulin spikes associated therewith.

It is another object of the invention to provide food compositions incorporating oral hypoglycemic agents of the invention, which are effective to reduce the glycemic index of food and blood glucose and insulin spikes associated therewith and which do not require expensive ingredient substitutions or pre-processing to reduce sugar or carbohydrate content.

It yet another object of the invention to provide a method of reducing the glycemic index of foods without requiring extensive adjustments in recipes or fomulations for food.

A further object of the invention is to provide a method for reducing blood glucose and insulin spikes associated with traditional high glycemic index food sources.

It is yet a further object of the invention to provide a method for consumers to self-initiate regimens that aid in reduction of blood glucose and insulin spikes associated with a diet high in sugars and simple carbohydrates and facilitate weight and fat loss.

Still another object of the invention is to provide dietary supplements and food additives comprising oral hypoglycemic agents in combination with other additives known to lower blood sugar or that have blood sugar homeostasis properties.

Other objects of the invention will be evident to one skilled in the art.

### SUMMARY OF THE INVENTION

Oral hypoglycemic agents as an additive to food stuffs will revolutionize treatment and prevention of diabetes mellitus and sequelae associated therewith by dramatically increasing compliance with a supplement or medication regimen. Further, addition of oral hypoglycemic agents to food stuffs facilitates weight loss and provides health benefits readily available to the population in general.

The invention is directed to food stuffs and dietary supplements incorporating oral hypoglycemic agents, methods for reducing the glycemic index of food stuffs by incorporating oral hypoglycemic agents, alone or in combination with other components, into food, and methods for reducing blood sugar and insulin spikes associated with high glycemic index foods. The invention is also directed to oral hypoglycemic agent supplements for use by consumers in the preparation of food stuffs in the home and in self-initiated supplement regimens. The oral hypoglycemic agents of the invention have not heretofore been used as food additives or with dietary supplements for the purposes of reducing the glycemic index of food or reducing blood sugar and insulin swings associated with consumption of high glycemic index foods, or for reducing weight or anti-aging.

In one embodiment, the invention is a food additive comprising at least one oral hypoglycemic agent alone or in combination with other oral hypoglycemic agents for use at the food manufacture or preparation stage or for self-initiated consumer regimens.

In another embodiment the invention is a food composition comprising the oral hypoglycemic agent food additives of the invention.

Another embodiment of the invention is a food additive consisting of metformin alone or in combination with other oral hypoglycemic agents for use at the food manufacture or preparation stage or for self-initiated consumer regimens.

In still another embodiment, the invention is directed to a method for altering the glycemic response to food comprising the step of: incorporating at least one oral hypoglycemic agent of the invention into the food during the manufacture or preparation process.

In yet another embodiment, the invention is directed to a method for maintaining blood sugar levels after food ingestion comprising the step of incorporating at least one oral hypoglycemic agent of the invention into the food during manufacture or other food preparation process.

Still another embodiment of the invention is directed to a method to reduce the glycemic index of food comprising incorporating at least one oral hypoglycemic agent of the invention into the food during manufacture or other food preparation processes, including without limitation at home preparation.

Other embodiments of the invention are directed to the use of oral hypoglycemic agents alone or in combination with dietary supplements in consumer self-initiated regimens.

Further embodiments of the invention are directed to a variety of food compositions containing the oral hypoglycemic agents of the invention, including without limitation solid and liquid food sources, such as baked goods, protein or other nutritional bars, nutritional shakes, powders for preparation of nutritional shakes, energy drinks, teas, nutritional supplements, and the like. Other food compositions suitable for the invention will be evident to one skilled in the art.

### DETAILED DESCRIPTION OF THE INVENTION

The invention is directed to food compositions that are fortified with one or more food additives comprising oral hypoglycemic agents, including without limitation prescription and non-prescription oral hypoglycemic agents not heretofore known or utilized as food additives or dietary supplements, wherein the additive is incorporated into the food at the manufacture or preparation stage. The invention is also directed to oral hypoglycemic agents in combination with dietary supplements or additives, including without limitation vitamins, minerals, trace elements, enzymes, nutraceuticals, nutrients, insulin mimetics and other supplements or additives, or other compounds known to be useful additives for reducing blood sugar or maintaining blood sugar homeostasis or deficient in diabetic patients. The invention is also directed to methods for reducing the glycemic index of food, reducing blood sugar and insulin spikes associated with food having a high glycemic index and allowing consumers to self-initiate regimens that aid in reducing blood sugar and insulin spikes in diets that are high in sugars and simple carbohydrates.

For purposes of this invention, “oral hypoglycemic agent” shall mean and include any compound or composition the effect of which is to lower the concentration of glucose (sugar) in the blood, regardless of mechanism, sensitize cells to insulin and/or reduce insulin swings after ingestion of food, whether prescription or non-prescription. The invention is not limited to those oral hypoglycemic agents used to treat diabetes, but also includes those oral hypoglycemic agents known to reduce blood sugar. According to the invention, oral hypoglycemic agents may be used alone or in combination with other oral hypoglycemic agents.

Use of the term “at least one” oral hypoglycemic agent shall mean and include oral hypoglycemic agents used alone or in combination. “Oral hypoglycemic agents” may be abbreviated as “OHA” or “OHA's” for purposes of the invention. “Oral hypoglycemic agents”, “OHA”, “OHA's” or “hypoglycemic agents” are used interchangeably herein.

“Food stuffs” or “food sources” or “food” or “food compositions” shall mean and include any solid or liquid food that may be ingested or consumed by healthy human subjects for nutritional or snack purposes, or by humans suffering from a variety of conditions, including without limitation diabetes, pre-diabetes, metabolic syndrome, obesity, high blood pressure, dyslipidemias, cancer, or other medical disorders. “Food stuffs”, “food sources”, “food compositions” and “food” are used interchangeably herein.

“Dietary additives” or “dietary supplements” shall mean and include vitamins, minerals, nutraceuticals, trace elements, enzymes, nutrients, or natural additives or supplements. Many of these additives or supplements compounds are also known to be useful for purposes of lowering blood glucose through a variety of mechanisms or maintaining blood sugar homeostasis. They are also commonly found to be deficient in diabetic patients. As such, they may be combined with the oral hypoglycemic agents of the invention .

“Sugars” and “starches” are a part of a large group of compounds known as “carbohydrates” and shall mean and include natural or processed simple sugars, i.e., monosaccharides containing single units of molecules, including but not limited to glucose, fructose, mannose and sucrose, or starches, i.e., polysaccharides, comprising long chains of single units of sugar molecules, and foods containing them. The terms “sugars”, “starches”, and “carbohydrates” may be used interchangeably herein and is not intended to be limiting of the invention.

Many oral hypoglycemic agents (“OHA's”) are known and have been studied extensively. By way of non-limiting example, the invention contemplates use biguanides (metformin), sulfonylureas (gliclazide, glimepiride, and glyburide), thiazolidinediones (TZD's, such as pioglitazone and rosiglitazone), alpha-glucosidase inhibitors (acarbose, miglitol), dipeptidyl-peptidase-4 (DPP-4) inhibitors (linagliptine, saxagliptine, sitagliptine, alogliptine), and sodium glucose cotransporter 2 (SGLT2) inhibitors (canagliflozine, dapagliflozine, empagliflozine, and ertugliflozine), alone or in combination, as food additives and dietary supplements. None of these oral hypoglycemic agents has heretofore been used as a food additive at the manufacture or preparation stage, or as food additives in self-initiated consumer regimens. While many of the oral hypoglycemic agents are prescription medications with FDA-approved uses, the invention is directed to expanding the uses of these agents to address public health and consumer needs, much like other prescription medications, such as proton pump inhibitors, antihistamines, and steroid inhalations to name a few, are now available to the public.

A well-known example of a prescription OHA is the biguanide metformin. Metformin is currently the most widely prescribed diabetes drug in the world, approved for use in Type 2 diabetes in in the United States in 1995. Metformin has natural origins from a plant, Galega officinalis, also known as goat's rue, French lilac, Italian fitch, or professor-weed. G. officinalis was found to be rich in guanidine, a substance with blood glucose-lowering activity that forms the chemical basis of metformin. This insulin sensitizing drug was first introduced in 1957 and is now made synthetically.

As a prescription medication, metformin is widely prescribed as a safe way to prevent or delay the development of diabetes. Data from the open-label Diabetes Prevention Program Outcomes Study (DPPOS) demonstrates that metformin is linked to a moderate but durable weight loss and is safe and well tolerated. Metformin has the added advantage of not causing hypoglycemic reactions associated with other prescription OHA's. According to the Diabetes Prevention Program Research Group, the pattern of metformin associated weight loss appears to differ from that observed with caloric restriction in that adipose (fat) tissue is affected more than lean tissue mass. Metformin may also mimic the effects of exercise. As a preventative, metformin is cost effective to reduce the financial burden of diabetes on the individual and the healthcare system.

Metformin is well studied and has many health benefits aside from delaying or preventing the onset of diabetes. As one example, metformin plays a role in delaying the aging process. Several mechanisms have been shown to delay the aging process, resulting in improved health span in animal models, including mammals. While not wishing to be bound by theory, these include: 1) caloric restriction; 2) alteration in GH/1GF1 pathways; 3) resveratrol (SIRT 1 activator); and 4) rapamycin (mTOR inhibitor). Metformin inhibits mTOR directly and indirectly via activating AMP-activated protein kinase. Metformin mimics a caloric restrictive state by inducing hypoglycemia and alters 1GF1 pathways.

The effect of metformin on aging has been extensively studied and has been associated with longevity in many rodent models. Metformin also extends the lifespan of nematodes, suggesting an evolutionarily conserved mechanism. A recent high impact study demonstrated that metformin reduces oxidative stress and inflammation and extends both lifespan and health span in a mouse model.

This notion led investigators to further study whether anti-aging effects can be demonstrated in the type 2 diabetes population. Notably, in the United Kingdom Prospective Diabetes Study (UKPDS), metformin, compared with other anti-diabetes drugs, demonstrated a decreased risk of cardiovascular disease. This has been suggested in other studies and meta-analyses and remains an active area of research.

In addition to anti-aging studies, numerous epidemiologic studies have shown an association of metformin use with a decreased risk of cancer, as well as decreased mortality. There is also evidence from studies performed both in-vitro and in-vivo of metformin's role in attenuating tumorigenesis. The mechanisms proposed relate to its effects on reducing insulin levels, improved insulin action, decreased IGF-1 signaling (central to mammalian longevity), as well as activation of AMP-kinase.

The decreased risk of cancer is based on the principle that reducing the amount of sugar available to feed cancer cells will significantly slow their growth. Metformin reduces blood sugar through a few different mechanisms: (1) decreasing the amount of sugar made by liver; (2) reducing the absorption of sugar from intestines; and (3) increases insulin uptake into healthy cells (decreasing sugar in the blood). As stated, metformin does this by activating AMPK (AMP-activated protein kinase), which plays an important role in insulin signaling, systemic energy balance, and metabolism of glucose and fats.

Activated AMPK slows cancer growth by two mechanisms: (1) reducing the amount of sugar available for cancer cells to consume, and (2) inhibiting an enzyme called mTOR (mammalian target of rapamycin) which is responsible for cell growth, including tumor cell growth. Metformin can also inhibit mTOR directly as well, independent of AMPK activation, slowing tumor growth. Metformin kills cancer stem cells which are thought to be the most resistant to chemotherapy and radiation treatments. Metformin also seems to prevent pre-cancerous cells from evolving into cancer cells, Metformin decreases the amount of circulating estrogen and testosterone, both of which can stimulate growth of hormone dependent tumors, breast and prostate cancer.

One of the most compelling human studies involving the use of metformin's anti-cancer activity comes from an analysis of 8,000 patients with type 2 diabetes who were followed for 10 years. It was found that there was a 54% lower risk of developing any type of cancer among those taking metformin compared with those not taking metformin.

In another study, the response rate to neoadjuvant chemotherapy (chemotherapy given before surgery to shrink the size of the tumor) among a group of patients with type 2 diabetes found that those taking metformin had a 24% chance of having a complete response (no residual cancer found during surgery) versus only an 8% chance of having a complete response if not taking metformin.

Other cancer risks may also be decreased. For example, the risk of developing colorectal cancer was found to be 200% greater for those with type 2 diabetes who were not taking metformin versus those taking metformin. And, survival was found to be 34% greater for those with type 2 diabetes who were taking metformin versus those that were not taking metformin.

In lung cancer patients, a study evaluating survival after chemotherapy shows that type 2 diabetics taking metformin had an overall survival rate of 20 months (and a progression free survival of 8.4 months) versus 13 months (and a progression free survival rate of 6.4 months) for type 2 diabetics not taking metformin.

Similarly, survival and recurrence after chemotherapy were studied among a group of ovarian cancer patients with type 2 diabetes. Those who used metformin had a 5-year overall survival rate of 63% versus only 23% in those patients not taking metformin. Progression free survival at 5 years (meaning the chance of being alive at 5 years with no evidence of cancer) was 51% in those taking metformin and 8% in those not taking metformin.

For prostate cancer, there was no risk reduction for developing prostate cancer seen among type 2 diabetics taking metformin compared with those not taking metformin. However, overall survival rate of those who have prostate cancer was 45% greater in type 2 diabetics taking metformin versus those not taking metformin. Yet another study found that metformin use improved survival by 24%.

Metformin is such a promising anti-cancer medication that there are approximately 150 clinical trials investigating its use in cancer treatment and prevention. Studies are ongoing in other areas as well. Given its potential for health benefits, along with reducing blood sugar and insulin swings associated with high carbohydrate and sugar diets, metformin is an ideal food additive, the benefits of which outweigh the risks. While metformin is a preferred oral hypoglycemic agent for addition to food stuffs, the invention is not limited to metformin. The invention contemplates use of a variety of oral hypoglycemic agents alone or in combination with other oral hypoglycemic agents or dietary supplements as food additives during the manufacture or preparation stage or for self-initiated consumer regimens. Preferably, combinations of agents having different mechanisms of action or antagonistic side effects should be employed together.

Not all of the oral hypoglycemic agents of the invention are prescription medications. An example of an over-the-counter or nonprescription OHA is berberine. Like metformin, berberine is also a plant-based product that has been used in Eastern Medicine for centuries, for which there are over 2,800 studies are available in PubMed. Berberine is an isoquinolone alkaloid and the active ingredient of Coptis chinensis, It may also be found in plants used in botanical medical practice including Goldenseal (Hydrastis canadensis), Oregon grape (Berberis aquifolium), and Barberry (Berberis vulgaris), Berberine's hypoglycemic effect is claimed to be similar to that of metformin in multiple studies. Berberine is also known to exert anti-cholesterol and antihypertensive effects. Berberine has not previously been used as a food additive during the manufacture or preparation stage of food.

Other agents known to have hypoglycemic effects or to be useful in blood sugar homeostasis are known in the art. Many of these agents not only have hypoglycemic effects but also many other health benefits. As such, they may be useful in combination with other oral hypoglycemic agents of the invention to achieve synergistic effects. By way of example, non-prescription supplements for use with the other oral hypoglycemic agents of the invention include but are not limited to cinnamon and derivatives thereof, resveratrol, jiaogulan (also known as Gynostemma pentaphyllum), gymnema, globe artichoke, various ginseng compounds and derivatives thereof, bitter melon, yerbe mate guarana damiana (YGD), gooseberry, banaba, huckleberry, nettle leaf, fenugreek, milk thistle and bilberry. Still other OHA's include tiliroside derivatives, Cinnamomum kanehirai, Antrodia camphorata, nicotinamide adenine dinucleotide (NAD+) and precursors thereof (such as nicotinamide riboside), and sirtuin-inhibiting compounds. One skilled in the art will be able to determine other suitable hypoglycemic agents for use in the invention.

Compounds having insulin mimetic properties are also especially useful in combination with the OHA's of the invention. Insulin mimetics act like insulin but do not build up fat. An example of an insulin mimetic is alpha lipoic acid (ALA), a potent antioxidant with reported cardiovascular benefits, which is available to consumers. Antidiabetic medicinal plants having insulin mimetic properties is described in an article by Patel et al., “An overview on antidiabetic medicinal plants having insulin mimetic property”, Asian Pacific J. Trop. Med., 2012; 2(4): 320-330, incorporated herein by reference. Other insulin mimetic additives useful in combination with OHA's will be evident to one skilled in the art.

Oral hypoglycemic agents are well known to have excellent benefits for both diabetic patient and non-diabetic patients. However, adherence to the use of these agents is limited due to accessibility, convenience and costs. The present invention is directed to resolving convenience and cost disadvantages by providing food compositions that incorporate OHA's as additives to reduce the glycemic index of food, facilitate better blood sugar control and reduce insulin spikes as compared to what would occur by ingesting food compositions that do not include such additives. Hence, the invention provides a way to protect against the onset of diabetes due to poor or unhealthy diets and the sequelae associated therewith. The invention also allows consumers to realize other significant health benefits associated with the use of oral hypoglycemic agents as described above, including without limitation, weight loss, fat reduction, anti-aging effects, and anti-cancer effects.

The invention eliminates the need for chemical alteration of sugars, starches or carbohydrates used in food stuffs. The invention does not require reformulating food stuffs as the OHA's of the invention may be utilized as low-level additives with no changes in recipes or other ingredients. The benefits of the use of the OHA's of the invention may thus be achieved without reduction of the nutritional or energy value of the other components of the food. The invention also eliminates the need for medications or supplements to alter digestion or GI motility to slow absorption of sugars and simple carbohydrates contained in food. The invention directly accomplishes the objective of reducing the glycemic index of food and reducing blood sugar and insulin spikes associate with high glycemic index foods through the addition of oral hypoglycemic agents at the food manufacture or preparation stage.

The invention contemplates incorporation of OHA's not only in food stuffs at the manufacture or preparation stage, but also in self-initiated consumer regimens. In such regimens, consumers may utilize the OHA's of the invention alone as a routine food additive or in combination with other daily or regular dietary supplements and/or with nutrients found to be deficient in diabetic patients. Such nutrient additives include but are not limited to zinc, manganese, magnesium, chromium, vitamin E, vitamin C, vitamin B12, chromium, biotin, Coenzyme Q10 and the like.

### Examples—Exemplary Uses

Exemplary uses are described but are not intended to be limiting of the invention.

The invention utilizes OHA's as additives to food during the manufacture or preparation stage or as dietary supplements. According to the invention, OHA's are directly added to food stuffs during preparation or production of food or incorporated into the diet by consumers as a food additive or in the form of a dietary supplement. There is no need to change formulations, recipes or ingredients of any food stuff when incorporating the OHA additives of the invention.

Incorporation of OHA's of the invention into food stuffs is done at different stages of the food manufacture or preparation process by a variety of ways known to one skilled in the food manufacture or preparation art. OHA's are mixed directly in dry (solid) or liquid components of a food stuff, dissolved and added at a later point in the food manufacture or preparation process, or applied directly to the surface of already prepared food.

Incorporation of OHA's of the invention into food stuffs is done in such a way to not denature proteins that may be present. In addition, non-baked goods such as protein bars that are cold-prepped or processed, and liquids such as protein and energy drinks have a pH that does not affect the OHA supplement adversely. Baked goods are preferably baked at lower temperatures over longer periods of time to inhibit denaturation of the proteins, which may adversely affect OHA efficacy.

Food stuffs that are suitable for incorporation of the OHA's of the invention include, but are not limited to, protein bars, cereal bars, granola bars, muffins, dough, flours, dry baking mixtures, baked goods, such as breads, rolls, cakes, cookies, brownies, and crackers, powdered shake and drink mixes, frozen goods, canned goods, dry mix goods, cereal and other grain products, such as ready to eat breakfast cereal, oatmeal, rices, and pastas, foods that are processed at low temperature, i.e., cold-processed, carbonated and non-carbonated beverages, and energy drinks, bottled water, teas, coffee, yogurt, fats and oils, such as butter, margarine, and other spreads, fruit and vegetable juice, sauces, milk, soy milk, almond milk, rice milk, high glycemic index snack foods, sugar packets, spices, condiments, and other food additives that increase a food's glycemic index.

As one example, a typical protein bar includes a high proportion of protein to carbohydrate/fat content. Protein bars include protein powder, such as whey ingredients, egg albumin, soy and casein, among others, flour, milk or other binders, complex carbohydrates, simple carbohydrates, sugar alcohols, fructose, dextrin, oils, fruit extracts, and flavoring agents, including but not limited to chocolate and other sugars. While protein bars are not typically viewed as having a high carbohydrate or sugar content, they are often used as meal replacements and are often claimed and viewed as healthier than they are. Incorporation of OHA's into protein bars reduces blood sugar and insulin swings associated with carbohydrate and sugar content, while without diminishing the nutritional value of the other ingredients

As another example, protein shakes or drinks contain protein sources, but may also include fruits, fats, dairy additives, fiber, and flavoring agents, which drive up the caloric content and glycemic effects. Protein shakes may also be added to other food stuffs for expediency to increase nutritional effect while ignoring the glycemic effect. Incorporation of OHA's into protein shakes lowers the glycemic effect without reducing the nutritional value of the shake or drink.

Energy drinks and nutritional drinks and shakes are often used to provide a boost in energy and as substitutes for meals. These drinks often have a high glycemic index due to their sugar content. Incorporating OHA's into energy and nutritional drinks at the manufacturing stage or consumption (ingestion) stage reduces their glycemic effects without lowering their energy value.

Consumers may reduce the glycemic value of a meal by choosing low glycemic foods over higher glycemic index foods, reducing the portion size of higher glycemic index foods or eating adding proteins or fats at the expense of carbohydrates that have a high nutritional value despite their glycemic index. While these strategies may be effective, in part, the food preparation process may be more time consuming and cumbersome, thus resulting in lower interest over time. An oral hypoglycemic agent food additive for incorporation into food by a consumer at the preparation step or at ingestion, reduces the amount of time that planning low glycemic index meals entails and avoids the elimination of high nutrient value carbohydrates from the diet while counteracting the negative effects of sugar in the body. OHA food additives of the invention deliver hypoglycemic medication and improve compliance with medication and dietary regimens,

The invention uses pharmaceutically or therapeutically effective amounts of OHA's in food stuffs or with dietary supplements to lower the glycemic index of the food and blood sugar and insulin swings associated with high glycemic index foods. A “pharmaceutically or therapeutically effective” amount is an amount sufficient to lower the glycemic index of the food composition or lower the glycemic response (blood sugar and insulin swings) to the food. Response to the OHA food additives of the invention may be assessed by routine blood sugar measurements utilizing a blood glucose meter or by following HgA1c over time, or by other blood glucose testing protocols known to one skilled in the art.

What amount is “pharmaceutically or therapeutically effective” may vary depending on a number of factors. The amount of OHA's used in any one food depends on the particular OHA selected, the potency of the OHA, typical dosing levels, side effects, frequency of ingestion of the food or supplement in which the OHA is incorporated, type and amounts of other ingredients, and the glycemic index of the food without OHA's. In general, the response of the consumer or patient to use of an OHA food additive also varies. It is well known that OHA's are titrated to response using traditional blood glucose testing protocols.

Therapeutic amounts of the OHA's of the invention are known. A typical prescription dose of metformin used in a diabetic patient ranges from 500 mg-1000 mg by mouth twice a day. FDA-approved doses of other prescription OHA's of the invention are known and depend on mechanism and potency of the particular OHA.

Use of the OHA's of the invention at prescription doses as food additives is within the scope of the invention; however, use of lower amounts of prescription OHA's is therapeutic based on the aforenoted factors and an assessment of response. The present invention is not limited to use of OHA's at prescription doses, particularly when used in the manufacture or preparation of foods that are ingested frequently. For food stuffs that are ingested frequently, OHA's are used in amounts ranging from about 10% to 50% of the prescription dose.

Use amounts for non-prescription hypoglycemic agents are known. Amounts of non-prescription hypoglycemic agents used in the inventive are the same as the doses (amounts) that are commercially available to consumers. As with prescription OHA's, the amount of non-prescription OHA's is used at typical dosing levels that are commercially available or in amounts ranging from about 10% to 50% of the typical dose depending on the frequency of use.

Exemplary dosing for non-prescription OHA's is known. By way of example, berberine is used as a food additive in the present invention in amounts ranging from 300 mg to 1500 mg. Use of berberine in amounts up to 1500 mg per day for weight loss have been reported. Alpha-lipoic acid is used as a food additive in amounts ranging from 100 mg to 1800 mg. Resveratrol amounts at a lower end of supplementation for cardiovascular health, insulin sensitivity, and longevity for somebody who is otherwise unhealthy is 5-10 mg daily. For persons who are otherwise healthy, dosages between the range of 150-500 mg are used. Doses of resveratrol commonly used range between 250-500 mg. Amounts of NAD or precursors thereof utilized in the food additives of the invention range from 5 mg to 500 mg.

OHA's are used in combination in the present invention to take advantage of synergistic hypoglycemic effects. In that event, the total amount of each individual OHA incorporated into food stuffs to lower glycemic effects of the food is reduced. A significant aspect of the invention is the combination of two OHA's that have antagonistic side effects. Antagonistic combinations are useful to counteract side effects of individual OHA's. As one example, metformin is known to cause diarrhea, and berberine is known to cause constipation. The combination of metformin and berberine offsets these side effects.

In accordance with the patent statutes, the best mode and preferred embodiment have been set forth, the scope of the invention is not limited thereto, but rather by the scope of the attached claims.

1. A food additive for lowering the glycemic index of a food composition or reducing the glycemic response to consumed food, comprising: at least one oral hypoglycemic agent present in an amount effective to lower the glycemic index of the food composition or reduce the glycemic response to the food,
wherein the oral hypoglycemic agent comprises a biguanide, a sulfonylurea, a thiazolidinedione, an alpha-glucosidase inhibitor, a dipeptidyl-peptidase-4 (DPP-4) inhibitor, or a sodium glucose cotransporter 2 (SGLT2) inhibitor, berberine, cinnamon and derivatives thereof, jiaogulan (Gynostemma pentaphyllum), gymnema, globe artichoke, ginseng compounds and derivatives thereof, bitter melon, yerbe mate guarana damiana (YGD), gooseberry, banaba, huckleberry, nettle leaf, fenugreek, milk thistle, bilberry, tiliroside derivatives, Cinnamomum kanehirai, Antrodia camphorata, nicotinamide adenine dinucleotide (NAD+) or sirtuin-inhibiting compounds, or mixtures thereof,
wherein the food additive is incorporated into the food composition at the manufacture or preparation stage or added to a food composition through a self-initiated consumer regimen,
wherein the additive eliminates the need to pre-treat a food composition to alter sugars, starches, or carbohydrates of food compositions chemically, and
wherein the additive eliminates or reduces the need for medications or supplements to alter digestion or GI motility to slow absorption of sugars and simple carbohydrates contained in food.

2. The food additive according to claim 1, wherein the at least one oral hypoglycemic agent is metformin, berberine or mixtures thereof.

3. The food additive according to claim 1, further comprising vitamins, minerals, enzymes, trace elements, insulin mimetics, nutraceuticals, nutrients, or mixtures thereof,
wherein the vitamins comprise vitamin E, vitamin C, vitamin B12, biotin, or mixtures thereof,
wherein the trace elements comprise zinc, manganese, magnesium, or chromium,
wherein the nutraceutical comprises Coenzyme Q10, and
wherein the insulin mimetics comprise alpha lipoic acid.

4. A method for altering the glycemic response to food comprising the step of: incorporating an effective amount of the food additive according to claim 1 into the food during the manufacturing or preparation process or at the time of consumption in a self-initiated consumer regimen.

5. The method of claim 4, wherein the at least one oral hypoglycemic agent is metformin, berberine or mixtures thereof.

6-14. (canceled)

15. The method according to claim 4, wherein the food additive is incorporated into a food comprising cold-processed foods, protein bars, granola bars, flours, dry baking mixes, baked goods, powdered shake and drink mixes, frozen goods, canned goods, dry mixes, pastas, rice, carbonated and non-carbonated beverages, juice, yogurt, milk, soy milk, almond milk, rice milk, bottled water, teas, coffees, high glycemic index snack foods, fats, oils, margarine, spreads, condiments, sugar packets, or spices.

16-17. (canceled)

18. A method of reducing the side effects associated with use of an oral hypoglycemic agent to reduce the glycemic index of food compositions comprising the step of: blending at least two oral hypoglycemic agents according to claim 1, wherein the oral hypoglycemic agents selected have antagonistic side effects to each other.

19. The method of claim 18, wherein the oral hypoglycemic agents consist of a mixture of metformin and berberine.

20. (canceled)

21. The food additive according to claim 1,
wherein the biguanide is metformin,
wherein the sulfonylurea is gliclazide, glimepiride, or glyburide,
wherein the thiazolidinedione is pioglitazone or rosiglitazone,
wherein the alpha-glucosidase inhibitor is acarbose or miglitol,
wherein the dipeptidyl-peptidase-4 (DPP-4) inhibitor is linagliptine, saxagliptine, sitagliptine, or alogliptine, and
wherein the sodium glucose cotransporter 2 (SGLT2) inhibitor is canagliflozine, dapagliflozine, empagliflozine, or ertugliflozine.

22. The food additive according to claim 2, further comprising nicotinamide adenine dinucleotide or precursors thereof, resveratrol, or alpha lipoic acid.

23. The food additive according to claim 22, further comprising zinc, manganese, magnesium, chromium, vitamin E, vitamin C, vitamin B12, chromium, biotin, or Coenzyme Q10.

24-26. (canceled)
